(19) United States
(12) Patent Application Publication
(10) Pub. No.: US 2006/0046279 A1
(43) Pub. Date: Mar. 2, 2006

(54) Analytical methods utilizing real-time energy/particle interaction-based determination techniques

(76) Inventor: Palestrina R. Truong, Boston, MA (US)

Correspondence Address: INTELLECTUAL PROPERTY / TECHNOLOGY LAW, PO BOX 14329, RESEARCH TRIANGLE PARK, NC, 27709, US

(21) Appl. No.: 10/925,308

(22) Filed: Aug. 24, 2004

(51) Int. Cl. C12Q 1/02

(52) U.S. Cl. 435/29

(57) ABSTRACT

Analytical methods utilizing energy/particle interaction assessment techniques, useful for monitoring and screening applications, including determinations of individuals suitable for inclusion in clinical trial test subjects, monitoring of the inception and progression of disease states, determinations of the character of drug/target interactions for drug discovery, determinations of best modes of therapeutic intervention in the treatment or prevention of disease and adverse physiological conditions, and monitoring of loci, e.g., environments including materials, food, air, etc., which are subject to presence or incursion of deleterious biological agents. The energy medium used in the energy/particle interaction can include laser energy, and the assessment technique can include the use of Electrophoretic Quasi Elastic Light Scattering (EQELS), Photon Correlation Spectroscopy (PCS) or Capillary Zone Electrophoresis (CZE).

### BACKGROUND OF THE INVENTION

1. Field of the Invention

This invention relates to analytical methods utilizing energy/particle interaction-based techniques, having application to a multiplicity of end uses, including, without limitation, longitudinal monitoring of patients during extended term therapeutic intervention, patient selection for clinical testing and treatment, selection of best mode treatments from potential alternatives for a given patient or patient group, design of drug development and biological synthesis efforts, and screening of materials and environments for the presence of deleterious chemical and/or biological agents.

2. Description of the Related Art

With the success of the Human Genome Project and contemporaneous developments in assay technology and high-throughput screening techniques, significant interest in the potential of “personalized medicine” has been generated. Personal medicine involves the application of comprehensive and integrated characterizing data of an individual, including individual bioindicators, disease states, physiological conditions, genetic predispositions, environmental exposures to various etiological agents, susceptibilities to infection, immune system profiles, receptor maps, etc., to determine the specific care and treatment of such individual. Such care and treatment may involve identification of specific therapeutic agents, doses, dose forms and dosing regimens, control of environmental exposure conditions, etc., based on the informational database for the patient.

Although the possibility of establishing individual biomarkers and rigorous genetic profiles has captured the imagination of those seeking new avenues of cost-effective healthcare, the promise of personal medicine has not materialized. There are various reasons for this circumstance, including cost considerations, lack of rapid diagnostic capability, non-availability of computational systems and software necessary for whole organism characterization, lack of reliable predictive models for therapeutically mediated responses for many disease states and physiological conditions, labor-intensive and/or time-consuming nature of many conventional assays, and entrenched preferences for treatment intervention rather than wellness or prevention.

Among the above-discussed impediments to personal medicine, the lack of rapid diagnostic capability is a major obstacle to progress.

In the field of drug development, patient populations utilized for clinical safety and efficacy studies typically exhibit a wide variation in individual susceptibility to side effects of the therapeutic agent being tested. The expense of drug development efforts could be substantially reduced by screening and selecting individuals who will respond to the drug without susceptibility to such side effects, with such screening and selection thereafter being employed in consumer usage of the drug to identify patients for whom such drug is beneficial without untoward side effects.

It is possible to perform screening for clinical testing and/or subsequent consumer use of the drug by nucleic acid diagnostic procedures that reveal differences in individual genetic makeup of different individuals. Such tests are time-consuming and costly. More importantly, however, these tests do not provide any information about a patient's response to such drug on a cellular level, a fact that is reflected in the 90% failure rate of the pharmaceutical industry's efforts to convert lead compounds into approved pharmaceuticals.

Thus, a technique is desirable that would obviate or at least effectively supplement nucleic acid-based testing approaches, and provide a better prediction of how a potential therapeutic compound will behave in a cellular environment. It would also be a significant benefit if such technique would provide insight into the defective character of diseased genes.

It would therefore be a significant advance in the art to provide means and method for achieving a rapid process determination and analysis of corporeal indicia of an individual.

In the field of biological analysis, much recent attention has been focused on methods of detection of biological agents used in terrorism activities. In particular, concern exists about the inability of conventional assay methods to provide rapid recognition of the presence of pathogenic species in locations of concern, such as water supplies, public gathering places, and air handling environments. The ability to provide rapid recognition of pathogenic species in such loci, with concomitant ability to rapidly select therapeutic and/or cidal agents for remedy of situations involving such pathogens, addresses a clear medical and security need.

A correlative need exists in infectious disease generally. Current infectious disease diagnostics provide qualitative determination of a patient's disease status within periods of time that may be from 48 hours to several days in duration. Such long determination times allow the pathogen when present in an individual to increase its presence and advance the extent and severity of the infection, before definitive identification is achieved and corresponding therapeutic intervention can begin. There is thus a compelling need in the healthcare field for an approach that will quickly provide empirical evidence to a physician or other healthcare provider to facilitate accurate diagnosis and correlative treatment producing improved patient outcomes.

The need for such rapid analytical capability in treatment of infectious disease is paralleled by the need for real-time analysis of microbial species in food industry applications, where microbial infections of food products pose health and safety risks, as well as pharmaceutical and biotechnology applications involving culturing and biological expressions and interactions.

In view of all of the foregoing, there is a substantial need in the art for rapid biological assays, for applications such as screening and validating drug targets in drug discovery efforts, preclinical development of drug compositions and formulations, clinical trial testing, and consumer use of approved drugs, for real-time identification of infectious disease and treatment thereof, for food industry and pharmaceutical/biotechnology manufacturing applications, and for bioterrorism counteraction involving monitoring and/or episodic assessment of environments susceptible to the presence or incursion of bioterror agents.

### SUMMARY OF THE INVENTION

The present invention relates to analytical methods utilizing energy/particle interaction assessment techniques, useful for monitoring and screening applications, including determinations of individuals suitable for inclusion in clinical trial test subjects, monitoring of the inception and progression of disease states, determinations of best modes of therapeutic intervention in the treatment or prevention of disease and adverse physiological conditions, and monitoring of loci, e.g., environments including materials, food, air, etc., which are subject to presence or incursion of deleterious biological agents.

Energy/particle interaction assessment techniques usefully employed in the broad practice of the present invention include, without limitation: Electrophoretic Quasi Elastic Light Scattering (hereafter “EQELS”); Photon Correlation Spectroscopy (hereafter “PCS;” also sometimes referred to as Dynamic Light Scattering (“DLS”) or as Quasi Elastic Light Scattering (“QELS”)); and Capillary Zone Electrophoresis (hereafter “CZE”).

In one aspect, the invention relates to an energy/particle interaction analysis method, including:

providing a sample including at least one particle from a source;

impinging on the sample an energy medium producing the energy/particle interaction;

assessing the energy/particle interaction using a technique selected from the group consisting of EQELS, PCS and CZE;

determining a quality of the source from assessment of the energy/particle interaction;

wherein the source is selected from the group consisting of (i) biological organisms and (ii) loci susceptible to presence or incursion of biologically deleterious agents, and

when the source is selected from (i) biological organisms, the quality is selected from the group consisting of:
(A) inception and/or progressionary character of a disease state or physiological condition during a period of time in which the inception or progression of the disease state or physiological condition mediates variation in energy interaction characteristics of said particle(s);
(B) suitability of individuals within a group of candidate biological organisms to constitute a class for therapeutic intervention, wherein said suitability is correlative with said energy/particle interaction for each of said individuals in said class of individuals;
(C) character of drug/target interaction involving an actual or potential therapeutic agent and a target derived from said biological organism;
(D) a best mode of therapeutic intervention selected from among a plurality of potential alternative therapeutic interventions; and
   when the source is selected from (ii) loci susceptible to presence or incursion of biologically deleterious agents, the quality of the source is its freedom from biologically deleterious agents therein.

Another aspect of the invention relates to a method of monitoring the inception and/or progressionary character of a disease state or physiological condition during a period of time in which the inception or progression of the disease state or physiological condition mediates variation in energy interaction characteristics of biological particles derived from a patient experiencing or susceptible to such disease state or physiological condition. Such method includes the steps of impinging on a sample including biological particle(s) from the patient, an energy medium producing an energy/particle interaction, and characterizing the energy/particle interaction by a technique selected from the group consisting of EQELS, PCS and CZE, with repetition thereof in a succession of samples derived from the patient at various times during the aforementioned period of time, and determining from corresponding energy/particle interactions and characterizations the inception and/or progressionary character of the disease state or physiological condition.

A further aspect of the invention relates to a method of screening a candidate population for clinical testing of a therapeutic agent to identify a study group of patients suited for therapeutic intervention using the agent, wherein the agent binds to a cellular receptor site whose presence is detectable by energetic interaction utilizing a detection technique selected from the group consisting of EQELS, PCS and CZE. The method includes the steps of obtaining a cellular sample from patients in the candidate group including cells of the type for which the therapeutic agent is potentially binding, and subjecting the patient samples to one or more of the techniques selected from the group consisting of EQELS, PCS and CZE, to produce an energy/cell interaction correlative of presence or absence of the cellular receptor. From the energy/cell interactions a patient group for said clinical testing is determined, as having the cellular receptor.

Yet another aspect of the invention relates to a method of therapeutic intervention for treatment of a patient having a cytologically presented characteristic indicative of a condition to which therapeutic interventions of varied type are varyingly effective. The method includes the steps of subjecting respective cellular samples from the patient to the variant therapeutic interventions, subjecting the samples to energy/cell interaction to characterize the cytologically presented characteristics of said cells in each of the therapeutic interventions, and determining from the energy/cell interactions a best mode of therapeutic intervention for treatment of the patient.

Other aspects, features and embodiments of the invention will be more fully apparent from the ensuing disclosure and appended claims.

### BRIEF DESCRIPTION OF THE DRAWINGS

FIG. 1 is a block diagram of an illustrative EQELS spectrometer that may be employed in carrying out methods in accordance with the present invention.

FIG. 2 is a block diagram of a specimen acquisition system of an illustrative type that may be employed in carrying out methods in accordance with the present invention.

FIG. 3 is a block diagram of an illustrative flow-through EQELS spectrometer.

FIG. 4 is a block diagram of an illustrative data processing system that may be usefully employed to carry out methods in accordance with the present invention.

FIG. 5 is a flow chart illustrating a method of screening a candidate population to determine a test group for clinical trials of a therapeutic agent.

FIG. 6 is a flow chart illustrating a method of monitoring the inception and/or progressionary character of a disease state or physiological condition.

FIG. 7 is a flow chart illustrating a method of therapeutic intervention for treatment of a patient having a cytologically presented characteristic indicative of a condition to which therapeutic interventions of varied type are varyingly effective.

FIG. 8 is a flow chart illustrating a drug discovery method conducted in accordance with one embodiment of the invention.

### DETAILED DESCRIPTION OF THE INVENTION, AND PREFERRED EMBODIMENTS THEREOF

The present invention contemplates the use for applications such as those described in the “Background of the Invention” section hereof, of rapid analysis by energy/particle interaction techniques.

While the invention is described hereinafter with primary reference to EQELS as the sensing and detection technique, it will be recognized that corresponding methodologies can be carried out with other modalities of energy/particle interactions, including, without limitation, CZE and PCS.

EQELS is a process for characterizing particles in an inhomogeneous particle-containing medium, which utilizes electrophoresis, in which particles are characterized by their movement in an applied electric field.

PCS involves particle-mediated scattering of light that is impinged on an inhomogeneous (particle-containing) medium and measurement of the temporal autocorrelation function for a scattering vector at a specific scattering angle. From scattering intensity and the autocorrelation function, one can determine particle size (hydrodynamic radii), shape factors and other characteristics of the particles in the particle-containing medium.

CZE involves flow of an inhomogeneous medium through a narrow tube with application of an electric field across the sample flowstream and detection of migration characteristics of particles in the sample under the applied field conditions.

The rapid analytical methods of the invention can be carried out utilizing EQELS, PCS and CZE techniques, or other suitable methods for detecting and/or characterizing particles, e.g., cells, microbes, binding pairs, etc., in which energy is impinged on a medium containing or susceptible to presence of the particles, to generate an energy interaction spectrum, and determining the presence, absence or character of such particles from the energy interaction spectrum.

The energy interaction spectrum generally can be of any suitable type, including energy scattering spectra, energy absorbance spectra, energy transmittance spectra, or any other spectrum indicative of the energy/particle interaction involving such species and/or agents. The energy interaction may be conducted under electrophoretic or non-electrophoretic conditions, and the energy source can be of any suitable type effective to generate the desired interaction spectrum, including, without limitation, electromagnetic energy, acoustic energy, ultrasonic energy, or any other suitable energetic medium.

In the case of electromagnetic energy, the energy can be of appropriate spectral regime, such as visible light, infrared, ultraviolet, and x-ray spectral regimes. In specific embodiments, actinic radiation is employed as the energetic medium for interaction with the particle in the sample, and such radiation can for example have a wavelength in a range of from about 200 nm to about 700 nm.

The rapid analysis techniques of the invention can employ visible light radiation, such as light-scattering techniques including classic light scattering and quasi-elastic light scattering.

Other embodiments employ uv radiation, such as capillary electrophoresis methods and systems having a uv laser as an energy source for uv radiation impinged on the particles in the capillary flow stream.

It will therefore be recognized that any suitable energy source and corresponding energy medium can be employed in the broad practice of the invention. In various preferred embodiments, a visible light laser is utilized as the energy source, for conducting EQELS, PCS or CZE techniques.

The rapid analysis methodology of the invention can be carried out under electrophoretic or non-electrophoretic conditions, as may be desired in a given application of the method.

In preferred practice, the rapid analysis methodology of the invention utilizes EQELS as a processing technique. In various applications, the EQELS methodology includes the steps of: impinging light on the sample to produce a scattered light output; and processing the scattered light output to determine (i) phase shift and Doppler shift of scattered light in the scattered light output, relative to the impinging light, and (ii) electrophoretic mobility of the particle(s) involved in the energy/particle interaction.

The EQELS methodology employed as an energy impingement and interaction technique in specific embodiments of the present invention has utility for rapid (typically less than 1 hour, and in many applications less than 5 minutes, e.g., less than 1 minute) detection and/or characterization of biological particles such as cells and/or microbes.

The EQELS methodology is suitable for use in detecting particles, which may comprise biological particles such as whole cells, living cells, dead cells, fixed cells, microbes, peptides, proteins, nucleic acids, polysaccharides, lipids, lipoproteins, microparticles, nanoparticles, metal, plastic, organic, ceramic, etc. Examples of specific particles to which EQELS techniques may be applied in embodiments of the invention include magnetic beads, glass beads, polystyrene beads, and the like. Such beads may serve as substrates or affinity media, and may be functionalized to provide ligands, surface binding sites, chemoattractive moieties, for binding, affiliation or association with particular species, or for other purposes. EQELS techniques may be employed in various applications within the broad scope of the present invention, e.g., to detect binding pairs of particles, one of which may be a target particle and the other of which may be a binder particle, with the respective particles specifically and selectively binding to one another.

Examples of binding pairs include, without limitation: cells and ligands; microbes and ligands; nucleic acid and nucleic acid; protein or peptide and nucleic acid; protein or peptide and protein or peptide; antigens and antibodies; receptors and ligands, haptens, or polysaccharides, complementary nucleic acids, pharmaceutical compounds, etc. Members of binding pairs may also be referred to as “binders.”

“Microbes” as used herein refers to viruses, bacteria, fungi and/or protozoa.

“Cells” as used herein refers to any types of cells, including human cells, animal cells (e.g., swine cells, rodent cells, canine cells, bovine cells, ovine cells, and/or equestrian cells) cloned cells, plant cells, etc., as well as cellular organelles, e.g., mitrochondria, Golgi apparatus, lysosomes, nucleoli, nuclei, or the like. The cells may be blood cells, cultured cells, biopsied cells, or cells that are fixed with a preservative. The cells can be nucleated, such as white blood cells or suspended endothelial cells, or non-nucleated, such as platelets or red blood cells.

The terms “a” and “an” as used herein include the singular as well as the plural, unless the context requires otherwise.

In various methods in accordance with the invention, EQELS can be used to determine identity, and presence or absence, in a solution, of a microbe, binding pair or other particle. EQELS is desirably employed as a laser spectroscopy technique for characterizing electrophoretic mobility behavior of particles in a sample.

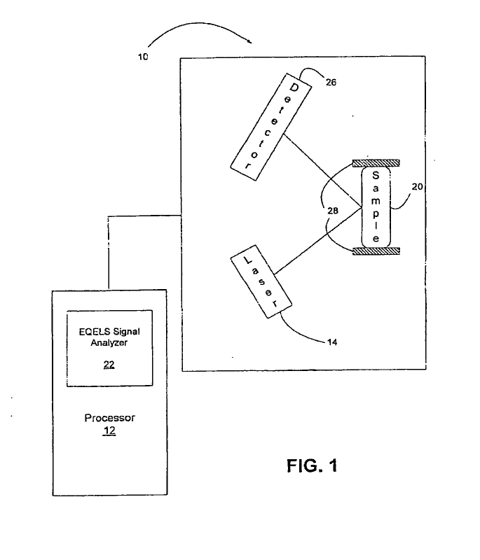

FIG. 1 is a block diagram of an illustrative EQELS spectrometer 10 that may be employed in carrying out methods in accordance with the present invention.

The spectrometer 10 includes a laser 14 that impinges a beam of light onto a sample 20. The sample 20 is positioned between two electrodes 28 that provide an electric field to the sample 20. Suspended, charged particles in the sample 20 are induced to move due to the application of the electric field. Movement of the suspended particles in the sample 20 is detected by quasi-elastic scattering from the generally coherent light provided by the laser 14. Some of the incident photons will encounter moving particles in the sample 20. When this encounter occurs, a small amount of energy from the photon is given up, and consequently, the frequency of the scattered light is slightly reduced. This scattered light is detected by a detector 26.

The spectrometer 10 is connected to a processor 12 that includes an EQELS signal analyzer 22. The processor 12 receives signals from the spectrometer 10, which are analyzed by the EQELS signal analyzer 22. For example, the scattered light detected by the detector 26 can be analyzed to determine the magnitude of the small shift in frequency. This shift in frequency is proportional to the rate of movement of the particle in the sample 20 and is detected as a Doppler shift. The signal analyzer 22 can measure the Doppler shift through a heterodyne technique in which unshifted light is mixed with the scattered light to produce “beats”. This signal is measured as an autocorrelation function that can then be Fourier transformed to yield a power spectrum for interpretation.

The EQELS spectrometer 10 can be used to detect and/or characterize biological cells and/or microbes, or alternatively other particles, in methods of various embodiments of the invention. For example, the EQELS spectrometer 10 can used to detect an EQELS spectrum for a sample 20 that includes a microbe in a solution. The EQELS spectrum is compared to a database of known spectra, each of the known spectra corresponding to one of a plurality of known microbes. The microbe in the solution is identified from the comparison.

As another illustrative example, the EQELS spectrometer 10 can be used to detect the presence or absence of a specific binding pair in a solution. A first EQELS spectrum of a solution including one member (e.g., the target) of the specific binding pair is detected. A specimen then is added to the solution and a subsequent EQELS spectrum is detected after adding the specimen. The EQELS spectra before and after the addition of the specimen are compared, and the presence or absence of the second member of the specific binding pair in the solution is detected based on the comparison.

As a still further illustrative example, the EQELS spectrometer 10 may be used to detect an EQELS spectrum for a sample 20 that includes a cellular specimen. This EQELS spectrum is compared to one or more known spectra of known cells. A characteristic of the cellular specimen can be assessed, such as diseases or abnormalities, including congenital, neoplastic or other conditions.

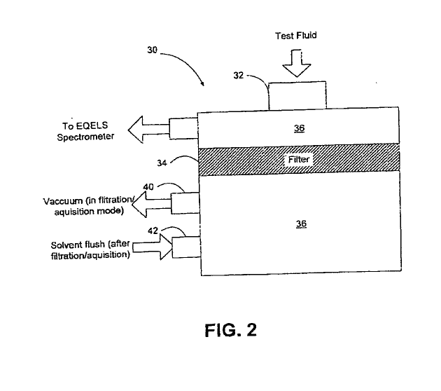

FIG. 2 is a block diagram of a specimen acquisition system 30 of an illustrative type that may be employed in carrying out methods in accordance with the present invention.

The sample acquisition system 30 includes an acquisition chamber 36 that includes a filter 34, inlets 32 and 42 and outlets 38 and 40. Valves (not shown) can be used to control flow between the inlets 32 and 42 or the outlets 38 and 40 and the chamber 36. In the FIG. 2 system, a vacuum can be provided in outlet 40 to create negative pressure in the chamber 36 so that test fluid enters the chamber 36 from the inlet 32. The test fluid can be a gas or liquid, such as air or water or other aqueous medium. The test fluid passes through the filter 34, and microbes and/or cells are filtered from the test fluid.

After a specimen is collected on the filter 34, a solvent enters the chamber 36 through the inlet 42. The solvent can combine with microbes and/or cells that have been collected on the filter to form a solution. The solution then exits the chamber through the outlet 38 to a collection area for subsequent EQELS spectroscopy or directly to an EQELS spectrometer. Although two inlets 32 and 42 and two outlets 38 and 40 are shown in FIG. 2 by way of illustration, it will be understood that other configurations can be employed, as necessary or desirable in a given application of the invention. For example, the outlet 40 and the inlet 42 can be combined to provide a single inlet outlet.

The acquisition system 30 in FIG. 2 can be used to automatically collect a sample for analysis from a fluid. For example, the acquisition system 30 could be miniaturized, automated and/or combined with an EQELS spectrometer and placed in various locations to monitor an air, water, and/or food supply. The acquisition system 30 can be used for bioterror surveillance to collect samples of fluids in an environment and/or monitor the collected samples for microbial agents. A telecommunications system can also be provided to communicate the results of the EQELS spectra obtained. When an EQELS spectrum is obtained that indicates the presence of a particular microbe is in the sample, a central command can be alerted through the telecommunications system and/or an alarm can be activated.

The acquisition system 30 can also be used to add various antibodies to a collected sample. For example, a pre-selected antibody with antigenic specificity against pathogens of bioterror significance could be added to a solution including the suspected microbe in the chamber 36, e.g., through the inlet 42. If the suspected microbe were present in the sample, the antibody may selectively modify the microbe's mobility. The change in mobility can be detected by a change in the EQELS spectra obtained before and after the addition of the antibody.

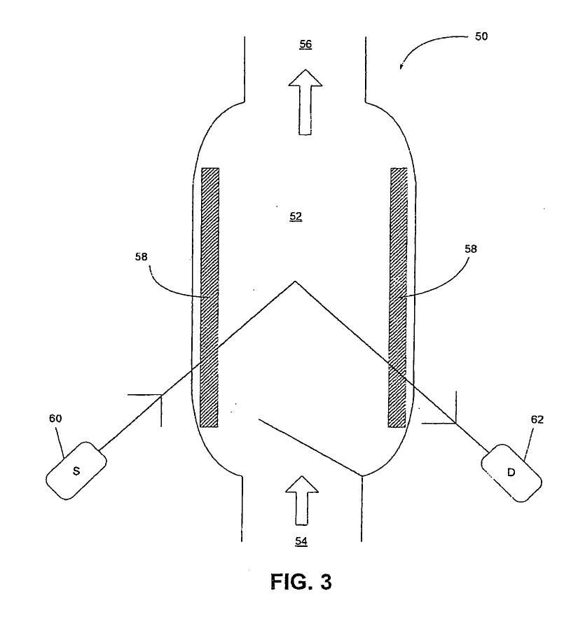

FIG. 3 is a block diagram of an illustrative flow-through EQELS spectrometer that can be employed to carry out methods in accordance with the invention, in various embodiments thereof.

The flow-through EQELS spectrometer illustrated in FIG. 3 utilizes a flow-through device 50 that includes inlet 54 and outlet 56 and a sample region 52 therebetween. The inlet 54 can include a valve (not shown in FIG. 3) for controlling the flow of a sample solution into the sample region 52. Electrodes 58 are positioned on opposite sides of the sample region 52 to produce an electric field. A light source 60 impinges a light beam on the sample region. The resulting scattered light is then detected by a detector 62.

The flow-through device 50 is arranged so that a sample solution including a microbe and/or cell of interest can flow into the sample region 52 through inlet 54. The inlet can be valved, and such valve can close when a suitable amount of sample solution has entered the sample region 52. The electrodes 58 produce an electric field in the sample region 52, and an EQELS spectrum is obtained using the incident light from the light source 60 and scattered light from the detector 62. The sample solution exits the sample region 52 through the outlet 56.

Although not shown for ease of illustration, a fluid pump, suction mechanism, and/or other techniques can be employed in the flow-thorough device to effect fluid removal from the sample region.

The outlet 56 can optionally be valved (not shown) for the purpose of controlling and directing fluid flow from the sample region 52. Another sample solution then can flow through the inlet 54 for subsequent testing. In this configuration, several sample solutions can be tested in rapid succession. In a specific arrangement, the flow-through device 50 can be connected to the acquisition system 30 shown in FIG. 2. It will be understood that other configurations of flow-through devices can be used in various embodiments of the present invention. For example, the inlet 54 and the outlet 56 can be replaced with a single opening to provide a combined inlet/outlet for batch-type operation.

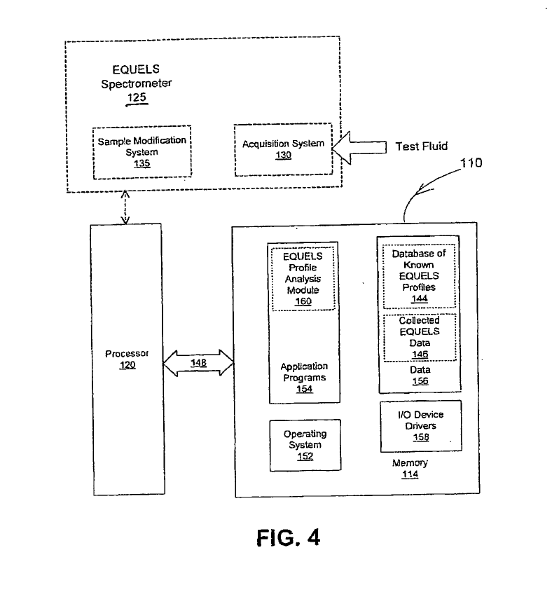

FIG. 4 is a block diagram of an illustrative data processing system 110 that may be usefully employed to carry out methods in accordance with the present invention.

The data processing system 110 includes a processor 120 in communication with an EQELS spectrometer 125, and a memory 114. Exemplary EQELS systems that can be used for the EQELS system 125 are illustrated in FIGS. 1 and 3.

The EQELS system 125 includes an acquisition system 130 and a sample modification system 135. The sample modification system 135 is configured to modify the sample in the spectrometer, such as by adding a substance, such as an antibody or a therapeutic agent, to the sample.

An illustrative acquisition system for acquiring a specimen for EQELS spectrometry is illustrated in FIG. 4. In some configurations, the EQELS spectrometer 125, the sample modification system 135 and/or the acquisition system 130 are omitted. For example, a sample can be positioned in an EQELS system 125 manually without requiring a separate acquisition system 130 and/or spectra can be obtained according to embodiments of the invention without modifying the sample with the sample modification system 135. In some embodiments, the EQELS spectrometer 125 is omitted and an EQELS spectrum obtained from a remote EQELS spectrometer is provided to the data processing system 110 for analysis.

The processor 120 communicates with the memory 114 via an address/data bus 148. The processor 120 can be any commercially available or custom microprocessor. The memory 114 is representative of the overall hierarchy of memory devices containing the software and data used to implement the functionality of the data processing system 110. The memory 114 can include, but is not limited to, the following types of devices: cache, ROM, PROM, EPROM, EEPROM, flash memory; SRAM, and DRAM.

As illustrated in FIG. 4, the memory 14 may include several categories of software and data used in the data processing system 110: the operating system 152; the application programs 154; the input/output (I/O) device drivers 158 and the data 156. The data 156 may include a database of known EQELS profiles 144 and/or EQELS data 146 from the EQELS system 125.

It will be appreciated that the operating system 152 can be of any suitable type for use with a data processing system. Illustrative examples of operating systems that can be usefully employed in the broad practice of the present invention include OS/2, AIX, OS/390 or System390 (International Business Machines Corporation, Armonk, N.Y.), Windows CE, Windows NT, Windows95, Windows98, Windows2000, or WindowsXP (Microsoft Corporation, Redmond, Wash.), Unix or Linux or FreeBSD, Palm OS from Palm, Inc., Mac OS (Apple Computer, Inc.), LabView or proprietary operating systems.

The I/O device drivers 158 typically include software routines accessed through the operating system 152 by the application programs 154 to communicate with devices such as I/O data port(s), data storage 156 and certain components of the memory 114 and/or the EQELS spectrometer 125.

The application programs 154 are illustrative of the programs that implement the various features of the data processing system 110 and can suitably include one or more applications that support analytical methods of the present invention. The data 156 represents the static and dynamic data used by the application programs 154, the operating system 152, the I/O device drivers 158, and other software programs that may reside in the memory 114.

While the EQELS profile analysis module 160 is illustratively constituted as an application program in FIG. 4, it will be appreciated that other configurations can also be usefully employed in carrying out the invention. For example, the EQELS profile analysis module 160 can also be incorporated in the operating system 152, the I/O device drivers 158 or other such logical division of the data processing system 110. Thus, any configuration capable of carrying out the operations for the methodology of the invention can be advantageously employed.

The I/O data port can be used to transfer information between the data processing system 110 and the EQELS spectrometer 125 or another computer system or a network (e.g., the Internet) or to other devices controlled by the processor.

In operation, an EQELS spectrometer such as the EQELS spectrometer 10 shown in FIG. 1 can be used to detect an EQELS spectrum for a sample, e.g., a sample that includes a microbe in a solution. The EQELS spectrum may be compared to a database of known spectra such that each of the known spectra corresponding corresponds to one of a plurality of known microbes. The microbe in the solution can be identified from the comparison. Microbes amenable to such analysis variously include viral, bacterial, fungal and protozoan microbes. Viral species can be of any suitable type, e.g., cytomegalovirus (CMV), herpes simplex virus (HSV), Epstein-Barr virus (HBV), respiratory syncytial virus (RSV), human immunodeficiency virus (HIV), etc.

The EQELS spectrum can be used to determine the electrophoretic mobility of a microbe, and the determined electrophoretic mobility can be used to identify the microbe. The electrophoretic mobility may depend on the surface charge of the microbe and/or on frictional forces resulting from the shape/size of the microbe and/or on the viscosity of the solvent. The surface charge on the microbe surface may also depend on solvent conditions such as pH.

In specific applications, the concentration of a microbe can be determined. The EQELS spectrum from a sample with an unknown microbial concentration can be compared with a spectrum from a sample with a known concentration. The integration of the spectrum (i.e., the area-under-the-curve) then can be used to determine the concentration.

The identification of the microbe can be facilitated in specific applications by addition of an antibody that binds to a specific microbe. When the antibody binds to the microbe, it can change both the surface charge and/or the frictional forces and thus, the antibody can change the electrophoretic mobility of the microbe. The electrophoretic mobility then can be determined from the EQELS spectrum.

The methodology of the invention can be utilized in specific applications to identify the presence or absence of bioterror agents, based for example on a specific list of potential microbial pathogens. A sample taken from a locus susceptible to the presence or incursion of a bioterror agent can be mixed with a cocktail mixture of antibodies against microbes-of-interest, and electrophoretic mobility of the cocktail-augmented sample can be determined from an EQELS spectrum and compared to an EQELS spectrum for the sample prior to addition of the antibody mixture, to determine any change in the profile of the sample indicative of the presence of a microbe of interest.

The methodology of the invention can also be employed to determine the sensitivity of a specific antibiotic or anti-microbial agent against a specific microbe. In order for an antibiotic or anti-microbial agent to exert a therapeutic effect, it must first bind to the surface of the microbe. When the antibiotic or anti-microbial agent binds to the surface, it can change the microbe surface charge and/or frictional forces. An EQELS spectrum or spectra can be used to detect such change.

If the antibiotic or anti-microbial agent produces either a cytostatic effect or a cidal effect on the microbe, a resulting change of the swimming rate of the microbe, its surface charge, and/or its volume (e.g., from swelling) is amenable to analysis. Thus, EQELS spectra can be used to determine whether an antibiotic or anti-microbial agent binds to a microbe and/or kills the microbe, and are useful to test a microbial sample for sensitivity to a particular antibiotic or anti-microbial agent. Accordingly, EQELS techniques can be employed in various embodiments of the invention in application to dead cells as the particles of interest, e.g., for cell death monitoring to assess the efficacy of therapeutic agents on or in the cells.

The binding constant for an anti-microbial agent can be determined from an EQELS spectrum of a sample including the microbe and the anti-microbial agent, to provide an indication of the effectiveness of the anti-microbial agent. For example, the concentration of the anti-microbial agent can be increased over time in the microbial sample solution. Changes in the mobility of the microbe as a function of the therapeutic agent concentration can then be determined from the EQELS spectrum. A resulting binding profile can be fitted to a binding model, such as a one-state binding model and/or a higher-state binding model, to provide a binding constant.

Parameters that can be used to identify microbes and/or to assess the effectiveness of an anti-microbial agent include swim rate (e.g., as determined by laser velocimetry), the ratio of the microbe swim rate to the electrophoretic mobility, the diffusion constant, the dimensions of the microbe (e.g., as determined by the diffusion constant and/or including radius of gyration, volume, characteristic dimension, structure factors, rod/cocci/axial ratios, etc.), and/or the ratio of a microbe dimension (e.g., its largest dimension) and the electrophoretic mobility.

Examples of fluids for which EQELS spectra can be obtained and various microbes in the sample assessed include, without limitation, blood, blood products, water, air, cerebrospinal fluid, ascites, pleural fluid, synovial fluid, etc.

In specific methods of the invention, the presence or absence of a specific binding pair in a solution is detectable by an EQELS spectrometer. An initial EQELS spectrum of a solution including one member of the specific binding pair (e.g., a cell) is detected. A specimen (in which the presence of the target species is to be determined) then is added to the solution and a subsequent EQELS spectrum is detected. The EQELS spectra before and after specimen addition are compared, and the presence or absence of the target species of the specific binding pair in the solution is detected based on the comparison. The target species of the specific binding pair in this example (in which a cell is the binding member) can include any ligand that binds to the cell surface, including chemical or biologic drugs and/or naturally occurring or synthetic substances, such as growth factors, hormones, lymphokines, chemokines, lipids, antibodies, biochemicals, etc. A change in the measured cell electrophoretic mobility can be detected based on the EQELS spectra taken before and after addition of the specimen, if specific binding has occurred.

In other methods of the invention, cellular specimens are analyzed utilizing an EQELS spectrometer arranged to detect an EQELS spectrum for a sample containing the cellular specimen. The EQELS spectrum then is compared to one or more previously determined spectra of known cells, to establish a possible match with one of the previously determined spectra, thereby enabling a characteristic of the cellular specimen to be assessed, such as a disease state or an abnormality (e.g., congenital, neoplastic or other condition).

Differences in electrophoretic mobility detectable by EQELS spectrometry can be used to detect abnormal cells, normal cell binding therapeutics or an abnormal ligand, and/or to provide detailed thermodynamic, biologic, clinical, and/or chemical information concerning cellular interaction. Examples of characteristics amenable to such analysis include, without limitation, binding constants, binding energies, binding specificity, and/or mapping of binding sites.

In a specific application, EQELS spectra can detect change in cellular electrophoretic mobility accompanying ligand binding. Ligand binding constants can be determined from the ligand concentration dependence of the cellular electrophoretic mobility change. Ligand binding constants are useful indices of ligand-cell interactions that can be related to biological efficacy of the ligand, mapping of a binding site, and/or the selection of a therapy.

For example, cells derived from a specific developmental cell line can express different surface epitopes. These differences can contribute to the identification of a cell, e.g., identification of the cell as a lymphocyte, granulocyte, T-cell, B-cell etc. Such cell surface epitopic variants produce different EQELS spectra that can be used to differentiate between respective cells, as for example between leukemic blasts and normal blood cells, between platelets and red blood cells, etc. The EQELS methodology of the present invention can detect these differences without the necessity of using fixed (preserved) cells, without incubation with a fluorescently labeled antibody, and/or without flow cytometry determinations.

In some cases, ligand binding to cells can lead to cell activation, such as occurs in thrombin (or other platelet agonists) binding by platelets or f-Met-Leu-Phe binding by neutrophilic granulocytes. Another example of cell activation is the activation of leukocytes. When a cell is activated, its surface changes to expose a different array of biologic molecules. These surface changes can result in a measurable difference in cell surface charge and therefore in electrophoretic mobility of the cell. When a cell dies, similar cell surface changes may occur, and may for example involve loss of electrochemical gradients across the cell membrane. These changes can be detected by changes in the EQELS spectra attributable to cell activation, cell death, etc.

Each type of tissue includes cells with unique surface characteristics. The uniqueness of the cell surface derives from expression of particular molecular species on the cell surface that permit the unique function and capability of each cell line. If a given cell binds a ligand to its surface or if the cell line becomes diseased, either through a congenital disease or an acquired disease, its cell surface will change. Such change of the cell surface can produce changes in the surface charges of the cell. An EQELS spectrum can be used to detect a change in the cell surface charge. The EQELS spectrum can also be used to detect specific drug binding, to detect the activation of enzymes on the cell surface, to distinguish normal cells from abnormal cells, to distinguish resting cells from activated cells, and/or to monitor drug efficacy and safety.

For any cellularly effective therapeutic agent to produce a useful therapeutic response, it must first bind to a targeted cell surface. The term “therapeutic agent” as used in such context includes any ligand or drug producing a desired therapeutic response. The avidity or strength with which a therapeutic agent binds to the cell often is the primary determinant of the usefulness or efficacy of the therapeutic agent. The interaction between the therapeutic agent and cell(s) can be assessed by EQELS techniques. Information obtainable from an EQELS spectrum by such techniques includes, without limitation, the natures of the biologic interaction, the chemical interaction, and/or the thermodynamic interaction between the therapeutic agent and a targeted cell surface. In addition to the determination of cellular binding of the therapeutic agent, the binding constant can be determined by EQELS techniques, as well as the factors that affect binding, such as the concentration of the therapeutic agent, temperature, the pH, the ionic strength, and the presence or absence of competing agents (including inhibitors of binding).

The differences in normal and abnormal cells can be detected using a comparison of EQELS spectra. For example, platelets with congenital abnormalities, such as Glantzmann's thromboesthinia or the Benard-Soulier syndrome, bind to certain ligands abnormally, and this abnormal binding may be detected by means of EQELS spectra generated before and after ligand addition to a platelet solution. Leukemic blasts can also be differentiated from normal blasts by comparing an EQELS spectrum of normal blasts to an EQELS spectrum of Leukemic blasts. The production of an abnormal product, such as a monoclonal antibody or a polyclonal antibody, is also detectable by EQELS techniques.

The effects of various types of therapeutic agents on a microbe and/or a cell can also be assessed by EQELS techniques. An EQELS spectrum can be generated for a sample before and/or after a therapeutic agent is administered. Therapeutic agents that may be assessed in this manner include, without limitation, drugs, hormonal agents, leukemic therapeutic agents, anti-platelet agents, pharmacological agents, vitamins, analytes and pH conditions.

Additionally, in respect of diagnosis, therapeutic intervention, patient monitoring, and other applications of the energy/particle interaction-based techniques in various embodiments of the present invention, the inventive methodology and systems can be used as an adjunct to conventional methods and systems, e.g., for corroboration, cross-correlation, enhancement of accuracy and reliability of diagnosis and interventional activity, etc. By way of example, an EQELS-based diagnostic procedure can be conducted in combination with a nucleic acid diagnostic procedure to screen prospective participants in a clinical testing program, or to assess whether a therapeutic agent should be administered, or to assess which of multiple possible drugs is most suitable for a specific individual. The EQELS-based diagnostic procedure in such applications can for example utilize cellular samples from a same tissue specimen from which cellular samples are derived for the nucleic acid diagnostic procedure.

EQELS-based methods of the present invention are also useful to evaluate, adjust and/or identify therapies in drug/treatment development programs and/or in clinical or pre-clinical drug trials or other drug development testing, including clinical or pre-clinical trials for developing or evaluating vitamin supplements, herbal remedies, and/or other treatments. EQELS-based methods of the invention also can be used to evaluate, adjust and/or identify a suitable dose of a selected treatment based on the effectiveness of the treatment as measured by EQELS spectra. Patient-specific assessments can be made to select appropriate therapeutic agents. The EQELS-based methodology in many cases obviates the need for reporter labels, and the EQELS process is non-destructive to the cells and ligands to which such process is applied.

In drug development applications of the EQELS methodology, large numbers of chemical structural variants of a basic molecular structure can be assessed to identify a lead compound with suitable binding affinity to a receptor or other target moiety. The EQELS methodology can also be used in the development of biologics, as well as generally in evaluating the efficacy of various compounds, including, without limitation, peptides, proteins, lipids, nucleic acids, and/or small molecules.

Examples of interactions that can be evaluated using one or more EQELS spectra include binding of coagulation factors to activated platelets, inhibition of platelet agonists, selective binding to cancer neoplastic tissue compared to normal tissue, surface activation, and enzyme interaction. The detection of interactions of a therapeutic agent with cells or microbes, and assessment of the biological, chemical and/or thermodynamic character of resulting binding, can be utilized to select therapeutic agents useful for specific treatment and/or disease-preventive applications.

The present invention contemplates an energy/particle interaction analysis method, including the steps of:

providing a sample including at least one particle from a source;

impinging on the sample an energy medium producing the energy/particle interaction;

assessing the energy/particle interaction using a technique selected from the group consisting of EQELS, PCS and CZE; and

determining a quality of the source from assessment of the energy/particle interaction;

wherein the source is selected from the group consisting of (i) biological organisms and (ii) loci susceptible to presence or incursion of biologically deleterious agents, and

when the source is selected from (i) biological organisms, the quality is selected from the group consisting of:
(A) inception and/or progressionary character of a disease state or physiological condition during a period of time in which the inception or progression of the disease state or physiological condition mediates variation in energy interaction characteristics of the particle(s);
(B) suitability of individuals within a group of candidate biological organisms to constitute a class for therapeutic intervention, wherein said suitability is correlative with said energy/particle interaction for each of said individuals in said class of individuals;
(C) character of drug/target interaction involving an actual or potential therapeutic agent and a target derived from the biological organism;
(D) a best mode of therapeutic intervention selected from among a plurality of potential alternative therapeutic interventions, e.g., wherein the best mode of therapeutic intervention is correlative with superiority of its energy/particle interaction in relation to energy/particle interactions of therapeutic interventions other than the best mode of therapeutic intervention in the plurality of potential alternative therapeutic interventions; and
   when the source is selected from (ii) loci susceptible to presence or incursion of biologically deleterious agents, the quality of the source is its freedom from biologically deleterious agents therein.

As used in such context, “character of drug/target interaction involving an actual or potential therapeutic agent and a target derived from the biological organism” is intended to be broadly inclusive of drug/target interaction characteristics, and associated causes and results of drug/target interaction, including, without limitation, drug discovery operations such as candidate drug screening, lead identification, lead validation, lead prioritization, lead optimization, target identification, target validation, target prioritization, pathway and mechanism studies, biosimulation and modeling of biological systems, etc.

The analytical methods of the invention, utilizing energy/particle interaction-based techniques, have application to a wide variety of end uses, including, without limitation, establishment of response rates of disease to single and/or combination drug therapy, establishment of safety and toxicity of single and/or combination drugs, establishment of pharmacokinetics of single and/or combination drug compositions, longitudinal monitoring of patients during extended term therapeutic intervention, patient selection for clinical testing and treatment, selection of best mode treatments from potential alternatives for a given patient or patient group, design of drug development and biological synthesis efforts, and screening of materials and environments for the presence of deleterious chemical and/or biological agents.

In one embodiment of the methodology of the invention, the source from which the particle is taken may be a biological organism, e.g., a human or veterinary (horse, sheep, cow, pig, etc.) subject, or a plant organism. The particle from the biological organism can be a cell, microbe, or other biological particle.

When the source from which the particle is taken to make up the sample includes a locus susceptible to the presence or incursion of biologically deleterious agents, such locus may include a structure, an environment, an aqueous medium, air, a land area, a material (e.g., a foodstuff or foodstuff precursor), articles (e.g., luggage or cargo), or any other thing, substance, or location in which the presence or amount of biologically deleterious agents may be of concern or interest.

The energy medium used in the methodology of the invention may include any suitable energetic medium, such as light, acoustic energy, ultrasound, or other forms of electromagnetic radiation or other energy. Light is a preferred energy medium for the practice of the methodology of the invention, and laser energy of suitable character may be employed, in a spectral regime appropriate to the specific application of the methodology, e.g., visible, ultraviolet, infrared, etc.

In specific applications of the methodology of the invention, the source may comprise a locus susceptible to presence or incursion of biologically deleterious agents, and the quality of the source from which the particle-containing sample is made up, may include freedom from biologically deleterious agents such as bioterrorism agents, e.g., agents such as sarin, mustard gas, anthrax (Bacillus anthracis), brucellosis (Brucella species), smallpox, West Nile virus, SARS virus, botulism toxin (Clostridium botulinum toxin), cholera (Vibrio cholerae), glanders (Burkholderia mallei), plague (Yersinia pestis), tularemia (Francisella tularensis), Q fever (Coxiella burnetii), filoviruses (e.g., Ebola, Marburg) and arenaviruses (e.g., Lassa, Machupo).

In other specific applications of the methodology of the invention, the source may include a biological organism, and the quality of the source to be assessed from the energy/particle interaction involving the sample may include inception and/or progressionary character of a disease state or physiological condition during a period of time in which the inception or progression of the disease state or physiological condition mediates variation in energy interaction characteristics of the particle(s) in the sample.

The disease state or physiological condition may include any of various states and/or conditions that are relevant to healthcare, wellness, disease prevention, amelioration, cure, etc. Examples include, without limitation, cancer, heart disease, viral infection (HIV and AIDS), osteoporosis, hypertension, atherosclerosis, diabetes, pulmonary hypertension, pulmonary diseases, renal diseases, connective tissue diseases, neurological diseases, and autoimmune conditions, cystic fibrosis, osteoporosis, etc.

The methodology of the invention in a specific implementation may be employed for development of drug or therapeutic biologicals, in cellular or microbial assays that can be performed in real-time to indicate whether a candidate drug or biological agent is efficacious for its intended purpose. Assays of the invention can thus be used for high-throughput screening of candidate therapeutic agents, to rapidly identify lead compounds or agents for further synthesis, derivatization, testing and development.

In still other specific applications of the methodology of the invention, the quality of the source to be assessed from the energy/particle interaction involving the sample may include suitability for therapeutic intervention of a class of individuals within the group of biological organisms, wherein such suitability is correlative with the energy/particle interaction for each of the individuals within such class of individuals. The class of individuals within the group of biological organisms may for example be human or other animal subjects that are selected for a clinical trial of a therapeutic agent, in which the methodology of the invention comprises selecting the clinical testing group and then conducting a clinical trial of the therapeutic agent using such class of individuals.

In yet other specific applications of the methodology of the invention, the quality of the source to be assessed from the energy/particle interaction involving the sample may include a best mode of therapeutic intervention selected from among a plurality of potential alternative therapeutic interventions, wherein the best mode of therapeutic intervention is correlative with superiority of its energy/particle interaction in relation to energy/particle interactions of therapeutic interventions other than the best mode of therapeutic intervention in the plurality of potential alternative therapeutic interventions.

In such best mode determinations for therapeutic intervention, the sample may include a cellular sample from the biological organism of interest, e.g., a human subject, a plant or other animal subject. For human or other animal subjects, the therapeutic intervention may include administration to the subject of a dose form of a specific medication, e.g., an oral dose form medication, a parenteral dose form medication, a transdermal dose form, or dose forms appropriate for any other suitable therapeutic agents involved in such determination. The therapeutic intervention may comprise interventions other than medicament administration, including radiological therapies, gene therapies (using suitable nucleic acid compositions, constructs, vectors, and administration modalities), physical therapies, etc.

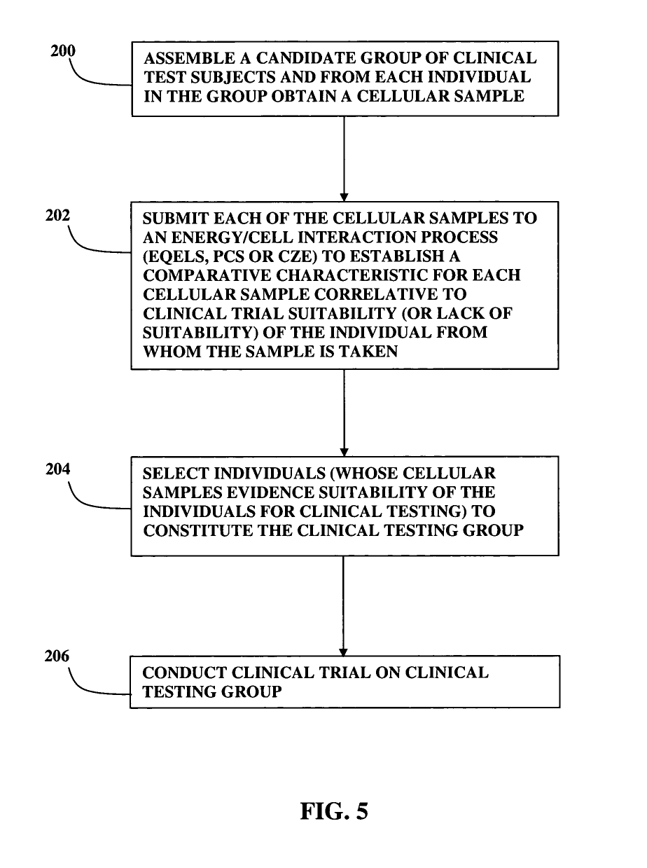

FIG. 5 is a flow chart illustrating one method of screening a candidate population to determine a test group for clinical trials of a therapeutic agent. In a first step 200, a group of candidate test subjects is assembled for the clinical testing, and a cellular sample is taken from each of the candidate individuals.

The second step 202 involves submitting each of the samples taken from the group of candidate testing subjects to an energy/cell interaction process, e.g., by EQELS, PCS or CZE, to establish a comparative characteristic for each cellular sample correlative to the clinical trial suitability or lack of suitability of the individual from whom the sample has been taken.

Next, in step 204, individuals are selected, whose cellular samples evidence suitability (in the energy/cell interaction-based determination) for clinical testing, with the selected individuals constituting the clinical testing group. Thereafter, in step 206, a clinical trial is conducted on such clinical testing group.

The methodology of the invention can be used in specific embodiments of the invention to monitor the inception and/or progressionary character of a disease state or physiological condition during an extended temporal period. Many diseases that originate in corporeal loci other than the blood-forming organs or their accessory tissue may nonetheless be significantly impacted by hemostasis. Examples include, without limitation, hypertension, atherosclerosis of blood vessels, diabetes, pulmonary hypertension, renal diseases, connective tissue diseases, infectious diseases, neurological diseases, and the like. Since nearly all bodily tissues are permeated by blood vessels, and the healthy states of such tissues depend on their perfusion by blood, abnormalities in the blood that affect hemostasis can lead to abnormal function of an organ or damage to an organ. For example, factors that either activate or damage endothelial tissue lining the blood vessels may induce the release of a number of substances from the endothelium, e.g., proteins, glycoproteins, lipoproteins, etc., with specific examples including von Willebrand factor, thrombomodulin, coagulation factor V, P-selectin, and the like. Other blood and cellular components, e.g., cytokines, lymphokines, calhedrins, chaperone proteins and the like, may be etiologically involved in endothelial activations or result from enthothelial activations. Identification of the presence of factors such as von Willebrand factor can enable early detection of disease, prognosis of the course of a disease, or a determination of the effectiveness of a therapeutic intervention intended to treat a disease.

The methodology of the present invention as applied to the detection of factors provides a significant diagnostic and monitoring tool enabling better understanding of disease states and physiological conditions, so that their etiology, prognosis and effective treatment can be elucidated.

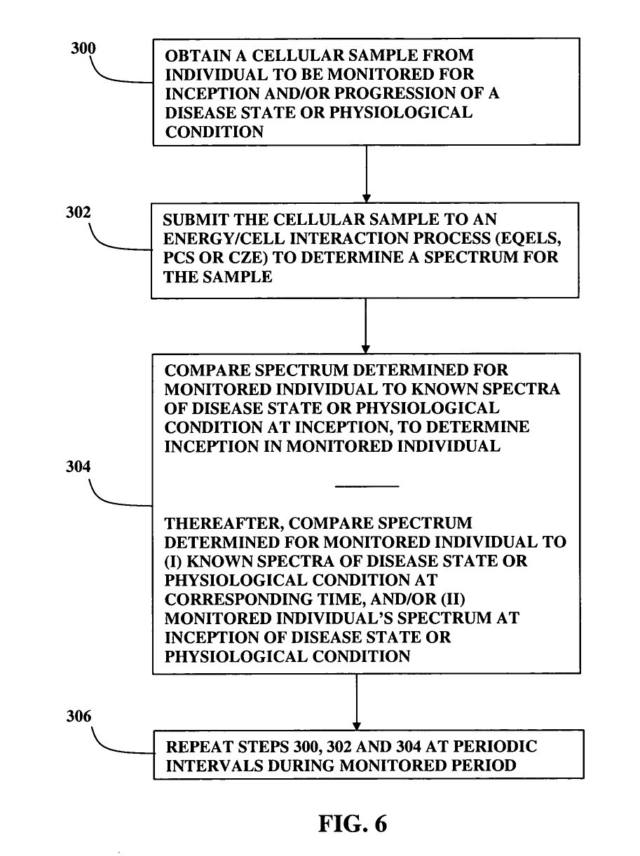

FIG. 6 is a flow chart illustrating a method of monitoring the inception and/or progressionary character of a disease state or physiological condition during a period of time (the monitoring period) in which the inception or progression of the disease state or physiological condition mediates variation in energy interaction characteristics of biological particles derived from a patient experiencing or susceptible to such disease state or physiological condition.

The method includes a first step 300 of obtaining a cellular sample from an individual to be monitored, followed by the second step 302 of submitting the cellular sample to an energy/cell interaction process, e.g., EQELS, PCS or CZE, to determine a spectrum for the sample.

Next, the step 304 is carried out, in which the spectrum determined for the sample in the second step 302 is compared to known spectra of cells with the disease state or physiological condition at inception, to determine if the sample from the monitored individual evidences the inception or post-inception development of the disease.

If inception is determined to have occurred, the sample spectrum is compared with known spectra of cells with the disease state or physiological condition in various stages of development, to determine the stage of development of the disease or condition in the individual, or if the time since inception of the disease or condition has been determined, then the sample spectrum is compared with known spectra of cells with the disease state or physiological condition at a corresponding time since inception, so that the progressionary status of the disease state or physiological condition can be assessed in relative terms (e.g., as being sub-normal in rate of progression, or as being supra-normal in rate of progression).

Additionally, or alternatively, the sample spectrum for a post-inception cellular sample can be compared with the spectrum of the monitored subject at the inception of the disease state or physiological condition, to determine a rate and/or extent of progression of the disease state or physiological condition, and/or the sample spectrum for a post-inception cellular sample can be compared with the spectrum for the cellular sample of the monitored subject at a prior time, or compared with various prior spectra for the monitored subject's earlier collected cellular samples, for the same purpose of determining a rate and/or extent of progression of the disease state or physiological condition.

As shown in step 306, the cellular sampling, spectral determinations and analysis steps are continued at periodic intervals during the monitoring period, which may for example in various embodiments be a period of days, weeks, months or years, as appropriate to the monitoring operation.

The above-described methodology may also be practiced in conjunction with the periodic administration of therapeutic agents (or administration of other therapeutic intervention) to the monitored individual during the period of monitoring, so that the efficacy of the therapeutic intervention during the monitoring period can be assessed, and the dosage regimen or other characteristic of the therapeutic intervention can be modulated as appropriate, to achieve an optimal therapeutic benefit to the monitored subject being treated by the therapeutic intervention, or the therapeutic intervention otherwise altered to the best interests of the patient.

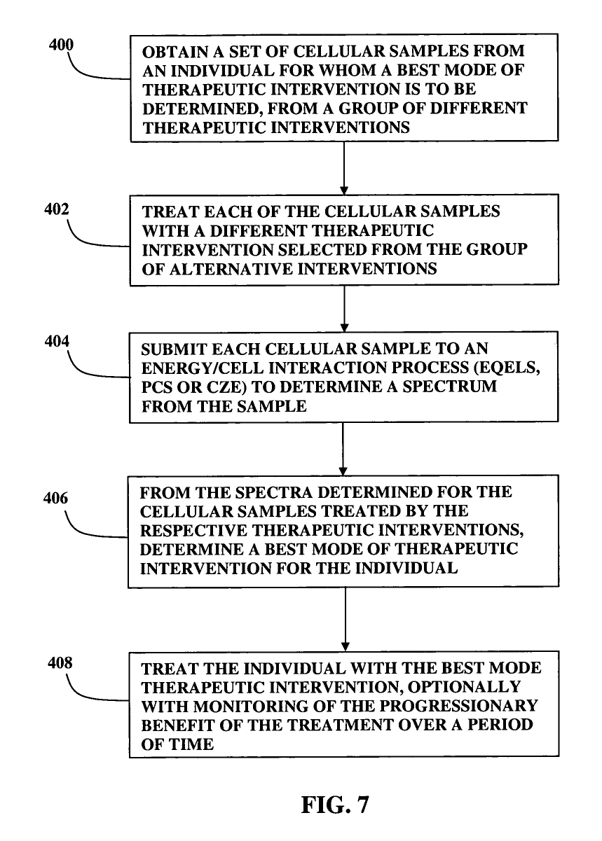

FIG. 7 is a flow chart illustrating a method of therapeutic intervention for treatment of a patient having a cytologically presented characteristic indicative of a condition to which therapeutic interventions of varied type are varyingly effective.

In step 400 of the method of FIG. 7, a set of cellular samples is obtained from an individual for whom a best mode of therapeutic intervention is to be determined from a group of differing alternative therapeutic approaches.

Each of the cellular samples thus obtained is treated with a different therapeutic intervention (selected from the group of potential alternatives) in step 402. After such treatment, each of the treated cellular samples is submitted to an energy/cell interaction process (EQELS, PCS or CZE) in step 404, to determine a spectrum for each sample.

Next, in step 406, from the spectra determined for the cellular samples treated by the respective therapeutic interventions, a best mode of therapeutic intervention is determined for the individual subject.

Finally, in step 408, the individual subject is treated with the best mode therapeutic intervention, optionally with monitoring of the progressionary benefit of the treatment over a period of time, as for example has been described hereinabove in connection with the discussion of the method depicted in FIG. 6.

In addition to the above-described applications, the energy/particle interaction-based techniques of the present invention may be utilized for drug discovery, including, without limitation, high throughput screening of drug candidates against a validated target for lead generation and optimization of potential therapeutic agents, as well as prioritization and validation of screened targets, target validation, pathway mapping and mechanism studies.

Such drug discovery applications may for example include cell-surface receptors, e.g., signaling receptors, adhesion receptors, transport receptors, etc., that interact with one or more therapeutic agents to produce a change, such as binding to a cognate ligand, producing a receptor conformational change, activating an intracellular biochemical response pathway, or inducing other cellular response, that is detectable by the energy/particle interaction-based technique (e.g., EQELS, PCS or CZP). Target validation and prioritization efforts may include comparison of targets based on their association with particular disease states or physiological conditions and the extent to which they regulate biological and chemical processes, and empirical verification that interactions of the therapeutic agent with the target correspond to desired change in the behavior of the associated cell.

The target may for example comprise a protein having a fundamental role in the onset or progression of disease. Once identified, libraries of potential drug compounds (leads) may be screened against the target to determine the leads that interact with the target with sufficient selectivity and effect to justify further testing and refinement as potential drug candidates.

The target may for example be present on a cellular surface, and the cells bearing the expressed target may be passed through an energy impingement and response monitoring cell in a system for carrying out EQELS, PCS or CZE in accordance with specific embodiments of the invention, with a specific lead candidate being contacted with the target in the monitoring cell and/or upstream thereof, to provide target/drug candidate interaction.

Once a selective and effective interaction is demonstrated for the target and the drug candidate, such lead may be submitted to lead optimization efforts. These efforts may for example involve synthesis of derivatives of the lead compound to refine the chemical structure and produce a drug candidate appropriate for preclinical and clinical testing. The lead optimization work may focus on various aspects of drug behavior and administration, including dosage concentration effects, selectivity for the target (greater selectivity being generally correlative with lower likelihood of adverse side effects), toxicological effects, pharmacokinetic behavior including duration of action and persistence in the body, and amenability to specific modes of administration (including formulation compatibility).

These various lead optimization efforts may likewise be carried out with energy/particle interaction techniques in accordance with various embodiments of the invention, to determine optimal drug agents for further study. For example, cells bearing the expressed target may be passed through an energy impingement and response monitoring cell in a system for carrying out EQELS, PCS or CZE in accordance with specific embodiments of the invention, with an optimized lead candidate being contacted with the target in the monitoring cell and/or upstream thereof, to provide target/optimized drug agent interaction.

The energy/particle interaction spectra generated during such drug development evaluations may be analytically processed to determine whether a specific target/drug interaction mediates a particular cellular response. For example, interaction of the target and drug may mediate intracellular processes that produce changes in cell size, conformation, epitopic artifacts, presence or absence of signaling proteins, etc. that alter the output of the energy/particle interaction and produce an energy interaction spectrum that is able to be compared with a database of spectra for the cells of interest. The database of spectra may for example include spectra for healthy cells, as well as spectra for cells at various stages of pathogenesis and/or remission. By algorithmic comparison of the spectrum generated by the energy/cell interaction after contact of the target with the drug agent, the nature of the corresponding target/drug interaction can be assessed.

In addition to the foregoing, the energy/particle interaction techniques of the invention may be used in various embodiments to explore pathway mapping in addition to target validation. Such mapping and validation determinations can employ EQELS, PCS and/or CZE techniques to exploit the study of signaling proteins, by allowing specific interactions to be studied in isolation.

Since the energy/particle interaction techniques of the invention enable rapid screening of chemicals against targets on a wide scale, such techniques can be carried out in various embodiments utilizing databases of known chemical and/or biological behavioral profiles for applications such as bio-simulation and modeling.

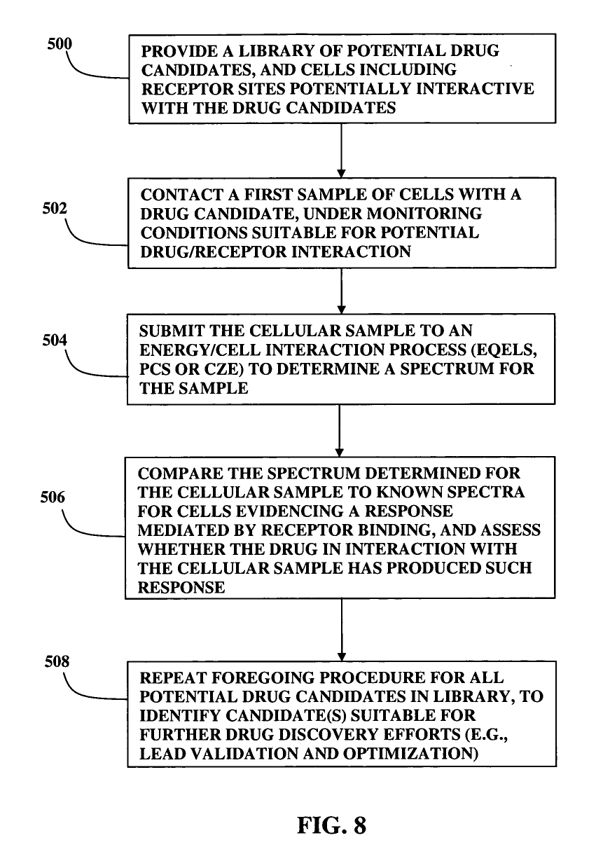

FIG. 8 is a flow chart illustrating a drug discovery method of screening a library of potential drug candidates against a target.

In step 500 of the method of FIG. 8, a library of potential drug candidates, and cells including receptor sites potentially interactive with the drug candidates, are assembled.

From the thus-provided library of potential drug candidates, a first candidate is contacted with a first sample of the cells, under monitoring conditions (i.e., conditions amenable to the subsequent energy/particle interaction processing) that are suitable for potential drug/receptor interaction (e.g., drug/receptor binding producing an agonistic or antagonistic effect, or otherwise affecting the activity of the receptor site), in step 502. This contacting may for example be immediately upstream of the monitoring cell (of the EQELS, PCS or CZE system), or the contacting may be carried out in such monitoring cell, or alternatively in a locus exterior to the EQELS, PCS or CZE system.

Next, in step 504, the cellular sample that has been contacted with the drug candidate is submitted to the energy/cell interaction process in the monitoring cell of the EQELS, PCS or CZE system, to produce a spectrum for the cellular sample.

Such spectrum for the cellular sample contacted with the drug candidate then in step 506 is compared to known spectra for cells evidencing a response mediated by receptor binding to assess whether the candidate drug in interaction with the cells in the cellular sample has produced such a response. If such a response has been generated by the interaction of the drug candidate, then the drug candidate becomes a lead for further drug discovery efforts. The database of known spectra may exist as a data structure in a processor/memory/spectrometer system of the type shown in FIG. 4 hereof.

The foregoing process of steps 502, 504 and 506 then is repeated in step 508 for each of the candidate drugs in the library, to identify candidate(s) suitable for further drug discovery efforts such as lead validation and optimization.

The foregoing procedure of FIG. 8 may be carried out in an analogous manner to assess different targets for target identification for a specific therapeutic agent (i.e., using a library of targets rather than a library of potential drug candidates). Additionally, lead validation or target validation may be carried out with energy/particle interaction-based assessments, employing techniques such as EQELS, PCS and/or CZE, in various embodiments of the invention.

In the practice of the methodology of the invention, the sample may be suitably obtained by any appropriate collection method that secures particle(s) from the source to be subjected to assessment. For spectral analysis, the particle(s) of the sample may be presented to the energetic medium for energy/particle interaction in an aqueous medium or carrier, or a suitable solvent or any other medium in which the energy/particle interaction can be effected.

It will be appreciated that the invention provides analytical methods utilizing energy/particle interaction-based techniques, having application to a multiplicity of end uses, such as longitudinal monitoring of patients during extended term therapeutic intervention, patient selection for clinical testing and treatment, selection of best mode treatments from potential alternatives for a given patient or patient group, design of drug development and biological synthesis efforts, and screening of materials and environments for the presence of deleterious chemical and/or biological agents.

Thus, while the invention has been variously described hereinabove with reference to specific aspects, features and embodiments, it will be recognized that the invention is not thus limited, but rather extends to and encompasses other variations, modifications and alternative embodiments, such as will suggest themselves to those of ordinary skill in the art based on the disclosure herein. Accordingly, the invention is intended to be broadly construed and interpreted, as encompassing all such variations, modifications and alternative embodiments, within the spirit and scope of the claims hereinafter set forth.

What is claimed is:

1. An energy/particle interaction analysis method, comprising:
providing a sample comprising at least one particle from a source;
impinging on said sample an energy medium producing said energy/particle interaction;
assessing the energy/particle interaction using a technique selected from the group consisting of EQELS, PCS and CZE; and
determining a quality of the source from assessment of the energy/particle interaction;
wherein said source is selected from the group consisting of (i) biological organisms and (ii) loci susceptible to presence or incursion of biologically deleterious agents, and when said source is selected from (i) biological organisms, said quality is selected from the group consisting of:
(A) inception and/or progressionary character of a disease state or physiological condition during a period of time in which the inception or progression of the disease state or physiological condition mediates variation in energy interaction characteristics of said particle(s);
(B) suitability of individuals within a group of candidate biological organisms to constitute a class for therapeutic intervention, wherein said suitability is correlative with said energy/particle interaction for each of said individuals in said class of individuals;
(C) character of drug/target interaction involving an actual or potential therapeutic agent and a target derived from said biological organism;
(D) a best mode of therapeutic intervention selected from among a plurality of potential alternative therapeutic interventions; and
and when said source is selected from (ii) loci susceptible to presence or incursion of biologically deleterious agents, the quality of the source is its freedom from biologically deleterious agents therein.

2. The method of claim 1, wherein the source comprises a biological organism.

3. The method of claim 2, wherein the particle from the source comprises a cell.

4. The method of claim 2, wherein the particle from the source comprises a microbe.

5. The method of claim 1, wherein the source comprises a locus susceptible to the presence or incursion of biologically deleterious agents.

6. The method of claim 5, wherein the locus comprises a structure.

7. The method of claim 5, wherein the locus comprises an air environment.

8. The method of claim 5, wherein the locus comprises an aqueous environment.

9. The method of claim 5, wherein the locus comprises a land area.

10. The method of claim 5, wherein the locus comprises a material.

11. The method of claim 10, wherein the material comprises a foodstuff or foodstuff precursor.

12. The method of claim 10, wherein the material comprises luggage.

13. The method of claim 10, wherein the material comprises cargo.

14. The method of claim 1, wherein the energy medium comprises laser energy.

15. The method of claim 1, wherein the energy medium comprises light.

16. The method of claim 1, wherein the source comprises a locus susceptible to presence or incursion of biologically deleterious agents, and the freedom from biologically deleterious agents comprises freedom from a bioterror agent.

17. The method of claim 16, wherein the bioterror agent comprises an agent selected from the group consisting of sarin, mustard gas, anthrax (Bacillus anthracis), brucellosis (Brucella species), smallpox, West Nile virus, SARS virus, botulism toxin (Clostridium botulinum toxin), cholera (Vibrio cholerae), glanders (Burkholderia mallei), plague (Yersinia pestis), tularemia (Francisella tularensis), Q fever (Coxiella burnetii), filoviruses and arenaviruses.

18. The method of claim 2, wherein the quality comprises inception and/or progressionary character of a disease state or physiological condition during a period of time in which the inception or progression of the disease state or physiological condition mediates variation in energy interaction characteristics of said particle(s).

19. The method of claim 18, wherein the disease state or physiological condition is selected from the group consisting of cancer, heart disease, viral infection, osteoporosis, hypertension, atherosclerosis, diabetes, pulmonary hypertension, pulmonary diseases, renal diseases, connective tissue diseases, neurological diseases, and autoimmune conditions.

20. The method of claim 18, wherein the disease state or physiological condition comprises cancer.

21. The method of claim 18, wherein the disease state or physiological condition comprises HIV infection or AIDS.

22. The method of claim 18, wherein the disease state or physiological condition comprises cystic fibrosis.

23. The method of claim 18, further comprising administering at least one therapeutic agent to said biological organism during said period of time, and monitoring therapeutic effect thereof during said period of time.

24. The method of claim 18, wherein the disease state or physiological condition comprises osteoporosis.

25. The method of claim 2, wherein the quality comprises suitability of individuals within a group of candidate biological organisms to constitute a class of individuals for therapeutic intervention, wherein said suitability is correlative with said energy/particle interaction for each of said individuals in said class of individuals.

26. The method of claim 25, wherein the class of individuals is selected for a clinical trial of a therapeutic agent.

27. The method of claim 26, further comprising conducting a clinical trial of said therapeutic agent using said class of individuals.

28. The method of claim 2, wherein the quality comprises character of drug/target interaction involving an actual or potential therapeutic agent and a target derived from said biological organism.

29. The method of claim 28, conducted as at least part of a drug discovery effort.

30. The method of claim 29, wherein said drug discovery effort comprises at least one operation selected from the group consisting of therapeutic agent screening, lead identification, lead validation, lead prioritization, lead optimization, target identification, target validation, target prioritization, pathway and mechanism studies, biosimulation and modeling of biological systems.

31. The method of claim 28, wherein character of the drug/target interaction includes selectivity of the therapeutic agent for the target.

32. The method of claim 28, wherein character of the drug/target interaction includes potency of the therapeutic agent in mediating a desired therapeutic effect.

33. The method of claim 28, wherein character of the drug/target interaction is determined for each of multiple therapeutic agents.

34. The method of claim 33, further comprising selecting from said multiple therapeutic agents a best one or best ones thereof according to comparative character of the drug/target interaction thereof, for drug development.

35. The method of claim 34, comprising therapeutic agent lead determination.

36. The method of claim 34, comprising therapeutic agent lead optimization.

37. The method of claim 28, comprising target validation.

38. The method of claim 28, comprising prioritizing targets.

39. The method of claim 28, comprising pathway mapping.

40. The method of claim 1, wherein said source is a human subject.

41. The method of claim 1, wherein said source is an animal subject.

42. The method of claim 1, wherein said source is a mammalian subject.

43. The method of claim 1, wherein said technique comprises EQELS.

44. The method of claim 1, wherein said technique comprises PCS.

45. The method of claim 1, wherein said technique comprises CZE.

46. The method of claim 1, further comprising making a comparative determination of said quality by an analytical process not involving said energy/particle interaction, for comparison with said quality determined from assessment of the energy/particle interaction.

47. The method of claim 2, wherein quality is selected from the group consisting of (B), (C) and (D).

48. The method of claim 47, further comprising making a comparative determination of said quality by a nucleic acid based analytical process, for comparison with said quality determined from assessment of the energy/particle interaction.

49. The method of claim 1, wherein said energy/particle interaction is conducted in a fluid medium.

50. The method of claim 49, wherein the fluid medium comprises an aqueous medium.

51. The method of claim 2, wherein the quality comprises a best mode of therapeutic intervention selected from among a plurality of potential alternative therapeutic interventions.

52. The method of claim 51, wherein said best mode of therapeutic intervention is correlative with superiority of its energy/particle interaction in relation to energy/particle interactions of therapeutic interventions other than said best mode of therapeutic intervention in said plurality of potential alternative therapeutic interventions

53. The method of claim 51, wherein said sample comprises a cellular sample from said biological organism.

54. The method of claim 53, wherein the biological organism comprises a human subject.

55. The method of claim 51, wherein the therapeutic intervention comprises administration to the biological organism of an oral dose form medication.

56. The method of claim 51, wherein the therapeutic intervention comprises administration to the biological organism of a parenteral dose form medication.

57. The method of claim 51, wherein the therapeutic intervention comprises administration to the biological organism of a gene therapy nucleic acid composition.

58. The method of claim 2, wherein the energy/particle interaction comprises a technique selected from the group consisting of EQELS and PCS.

59. The method of claim 2, wherein the energy/particle interaction comprises a PCS technique.

60. The method of claim 2, wherein the energy/particle interaction comprises an EQELS technique.

61. The method of claim 2, wherein energy/particle interaction comprises a CZE technique.

62. A method of monitoring the inception and/or progressionary character of a disease state or physiological condition during a period of time in which the inception or progression of the disease state or physiological condition mediates variation in energy interaction characteristics of biological particles derived from a patient experiencing or susceptible to such disease state or physiological condition, said method comprising impinging on a sample including biological particle(s) from said patient, an energy medium producing an energy/particle interaction, and characterizing said energy/particle interaction by a technique selected from the group consisting of EQELS, PCS and CZE, with repetition thereof in a succession of samples derived from said patient at various times during said period of time, and determining from corresponding energy/particle interactions and characterizations the inception and/or progressionary character of the disease state or physiological condition.

63. The method of claim 62, wherein said patient is being subjected to therapeutic intervention for treatment or prevention of the disease state or physiological condition during said period of time.

64. The method of claim 63, further comprising determination of the therapeutic efficacy of the therapeutic intervention.

65. The method of claim 63, wherein the therapeutic intervention comprises administration to the patient of a therapeutic agent.

66. The method of claim 62, wherein said technique comprises EQELS.

67. A method of screening a candidate population for clinical testing of a therapeutic agent to identify a study group of patients suited for therapeutic intervention using said agent, wherein said agent binds to a cellular receptor site whose presence is detectable by energetic interaction utilizing a detection technique selected from the group consisting of EQELS, PCS and CZE, said method comprising obtaining a cellular sample from patients in said candidate group including cells of the type for which the therapeutic agent is potentially binding, and subjecting said patient samples to said techniques selected from the group consisting of EQELS, PCS and CZE, to produce an energy/cell interaction correlative of presence or absence of said cellular receptor, and determining from said energy/cell interactions a patient group for said clinical testing, as having said cellular receptor.

68. The method of claim 67, wherein said technique comprises EQELS.

69. A method of therapeutic intervention for treatment of a patient having a cytologically presented characteristic indicative of a condition to which therapeutic interventions of varied type are varyingly effective, comprising subjecting respective cellular samples from said patient to the variant therapeutic interventions, and subjecting said samples to energy/cell interaction to characterize the cytologically presented characteristics of said cells in each of said therapeutic interventions, and determining from the energy/cell interactions a best mode of therapeutic intervention for treatment of the patient.

70. The method of claim 69, wherein said therapeutic interventions of varied type comprise different therapeutic agents.

71. The method of claim 69, wherein said therapeutic interventions of varied type comprise different dosages of a same therapeutic agent.

72. The method of claim 69, comprising EQELS technique conducted with laser energy as the energy of said energy/cell interactions.
